(19) United States
(12) Patent Application Publication
(10) Pub. No.: US 2009/0156581 A1
(43) Pub. Date: Jun. 18, 2009

(54) Aminergic pharmaceutical compositions and methods

(75) Inventors: Patrick F. Dillon, East Lansing, MI (US); Robert S. Root-Bernstein, East Lansing, MI (US)

Correspondence Address: HARNESS, DICKEY & PIERCE, P.L.C., P.O. BOX 828, BLOOMFIELD HILLS, MI, 48303, US

(73) Assignee: BOARD OF TRUSTREES OF MICHIGAN STATE UNIVERSITY, East Lansing, MI (US)

(21) Appl. No.: 11/918,549

(22) PCT Filed: Apr. 14, 2006
(86) PCT No.: PCT/US2006/014165

§ 371 Date: Oct. 15, 2007

Related U.S. Application Data
(60) Provisional application No. 60/672,224, filed on Apr. 15, 2005.
(60) Provisional application No. 60/706,249, filed on Aug. 5, 2005.
(60) Provisional application No. 60/738,294, filed on Nov. 18, 2005.

(51) Int. Cl. A61K 31/375; A61K 31/473; A61K 31/485; A61K 31/445; A61K 31/439; A61K 31/451; A61K 31/197; A61K 31/185; A61K 31/437; A61K 31/48; A61K 31/55

(52) U.S. Cl. 514/217.02; 514/474; 514/284; 514/282; 514/315; 514/289; 514/327; 514/562; 514/665; 514/293; 514/292

(57) ABSTRACT

Pharmaceutical compositions and method using aminergic compounds and complement compounds. Compositions are provided comprising: (a) a subefficacious amount of a non-adrenergic aminergic compound or of an adrenergic antagonist; and (b) a safe and effective amount of a complement compound. Methods are also provided comprising the administration of: (a) a low dose of a non-adrenergic aminergic compound or of any adrenergic antagonist; and (b) a safe and effective amount of a complement compound. Non-adrenergic aminergic compounds can comprise a histaminergic, dopaminergic, muscarinergic, serotoninergic, octopaminergic, or trace aminergic compound. Complement compounds include ascorbates, opioids, polycarboxylic acid chelators, resveratrols, cysteines, substituted derivatives and analogs thereof, and mixtures thereof. Preferred complements include ascorbates, particularly ascorbic acid. Methods include the treatment of: neurological and neural disorders; mood and behavior disorders; cardiac, vascular, and cardiovascular disorders; hypertension, headache; respiratory disorders; gastrointestinal disorders; obesity; asthma, allergy; smooth muscle contraction disorders; nasal or nasopharyngeal conditions; genitourinary disorders; ocular disorders, glaucoma; hormone- or neurotransmitter-release or -secretion disorders.

### CROSS-REFERENCE TO RELATED APPLICATIONS

The present application claims priority under 35 U.S.C. §119 to U.S. Provisional Application Ser. No. 60/672,224, filed Apr. 15, 2005, and to U.S. Provisional Application Ser. No. 60/706,249, filed Aug. 5, 2005, and to U.S. Provisional Application Ser. No. 60/738,294, filed Nov. 18, 2005. The disclosures of the above applications are incorporated herein by reference.

### BACKGROUND

The statements in this section merely provide background information related to the present disclosure and may not constitute prior art.

This invention relates to novel methods of treating disorders mediated by aminergic G-Protein-Coupled Receptors (GPCRs), and novel pharmaceutical compositions containing aminergic compounds. For example, the compositions and methods of this invention comprise the use of aminergics and ascorbates in the treatment of a variety of disorders,

Biogenic amines are involved in the regulation of a wide variety of body functions. Aminergic compounds include native biogenic amines, derivatives and synthetic analogs thereof, as well as entirely synthetic compounds having biogenic amine GPCR receptor-affecting activity. For example, aminergic compounds have their effect directly or indirectly on the alpha- and beta-adrenergic receptors, which are located throughout the body and mediate conditions, and treatments therefor, including hypertension, cardiac arrhythmia, heart failure, allergy and asthma, shock, and anaphylaxis. Similarly, other (non-adrenergic) aminergic compounds exert their effects by directly or indirectly binding to members of the other major biogenic amine receptor families: the histamine, dopamine, muscarinic acetylcholine, serotonin, octopamine, and trace amine receptors.

Receptors from among the non-adrenergic families are also broadly involved in a variety of diseases, disorders, and conditions, examples of which include: Parkinson's disease and movement disorders (e.g., dyskinesia); seizure or vomiting disorders; bipolar illness, schizophrenia, and other psychoses; other CNS diseases and disorders; depression and panic disorder; obsessive-compulsive disorders, bulimia and binge eating disorder; addictions and pleasure responses; obesity; learning, memory, and cognitive dysfunctions; neurovascular disorders and migraines; acute and chronic pain; hormone and neurotransmitter release disorders; lacrimal, salivary, and gastric secretion disorders; asthma, allergies, and inflammation; and parasympathomimetic disorders, e.g., related to intestine, bladder, and other smooth muscle contractions; among others. These receptors can similarly be utilized to mediate treatments therefor.

However, as is the case for administered aminergic compounds generally, non-adrenergic aminergic compounds administered for treatments are usually provided systemically. Normally, only one condition necessitates the treatment; yet, because adrenergics, and non-adrenergic aminergics, exert their effects broadly on their respective receptors throughout the body, other effects are induced non-specifically. As a result, these compounds have been found to exhibit undesirable side-effects. This has made selection of an adequate, but not excessive, dose level to be an exacting determination. As a result, it would be desirable to provide treatments, mediated through non-adrenergic aminergic GPCRs that exhibit a reduction in side-effects and/or in which a smaller dose of aminergic compound were used; as well as to provide treatments, mediated through adrenergic GPCR antagonism, that exhibit a reduction in side-effects and/or in which a smaller dose of an adrenergic antagonist compound were used.

### SUMMARY

The present invention provides pharmaceutical compositions comprising aminergic compounds and complement compounds. The complement compound(s) enhance the degree or duration of effect of the aminergic compound and can thus be used to enhance the effect of a given administered dose of aminergic, or for a given level of effect, can permit use of a decreased dose of aminergic. Embodiments of this invention include compositions comprising:
(a) a subefficacious amount of a non-adrenergic aminergic compound or of an adrenergic antagonist compound; and
(b) a safe and effective amount of a complement compound.
   Other embodiments include compositions comprising:
(a) a safe and effective amount of a non-adrenergic aminergic compound or of an adrenergic antagonist compound; and
(b) a complement compound selected from the group consisting of hyperpreserving amounts of ascorbates, analogs and substituted derivatives thereof; safe and effective amounts of opiates, analogs and substituted derivatives thereof; hyperpreserving amounts of polycarboxylic acid chelators, analogs and substituted derivatives thereof; hyperpreserving amounts of resveratrol family members, analogs and substituted derivatives thereof; safe and effective amounts of cysteine family members, analogs and substituted derivatives thereof; and mixtures thereof.

Methods are also provided for regulating an adrenergic, histaminergic, dopaminergic, muscarinergic, serotoninergic, octopaminergic, or trace aminergic receptor in a human or animal subject, comprising the administration of:
(a) a low dose of a non-adrenergic aminergic compound or of an adrenergic antagonist compound; and
(b) safe and effective amount of a complement compound.

Preferably, the non-adrenergic aminergic compound comprises a histaminergic, dopaminergic, muscarinergic, serotoninergic, octopaminergic, or trace aminergic compound; preferably, the aminergic compound is a histaminergic, dopaminergic, muscarinergic, or serotoninergic compound. Preferred complements include ascorbates, particularly ascorbic acid. Methods include the treatment of neurological and neural disorders; mood and behavior disorders; cardiac, vascular, and cardiovascular disorders; hypertension, headache; respiratory disorders; gastrointestinal disorders; obesity; asthma, allergy; smooth muscle contraction disorders; nasal or nasopharyngeal conditions; genitourinary disorders; ocular disorders, glaucoma; hormone- or neurotransmitter-release or -secretion disorders.

It has been found that the compositions and methods of this invention are effective for treating a broad range of disorders associated with non-adrenergic biogenic amine receptors. Use of these methods and compositions afford advantages versus non-adrenergic aminergic compositions and methods among those known in the art, including enhanced efficacy, increase duration of action, reduction of side effects, and dosing flexibility.

Further areas of applicability will become apparent from the description provided herein. It should be understood that the description and specific examples are intended for purposes of illustration only and are not intended to limit the scope of the present disclosure.

### DRAWINGS

The drawings described herein are for illustration purposes only and are not intended to limit the scope of the present disclosure in any way.

The present invention will become more fully understood from the detailed description and the accompanying drawings, wherein:

FIG. 1 presents spectrograms demonstrating binding of ascorbate to the human H1 histamine receptor (HR) in in vitro suspensions: =0 μg/mL HR without Ascorbate, ◯=0 μg/mL HR with Ascorbate, ▪=3.1 μg/mL HR without Ascorbate, □=3.1 μg/mL HR with Ascorbate, ▴=9.4 μg/mL HR without Ascorbate, Δ=9.4 μg/mL HR with Ascorbate, ♦=31.4 μg/mL HR without Ascorbate, ⋄=31.4 μg/mL HR with Ascorbate;

FIG. 2A-2C presents graphs showing the effect of absolute and relative human H1 histamine receptor (HR) concentrations on the rates of Asc oxidation and reduction; in FIG. 10A: =0 nM HR, ▴=56 nm HR, ▪=170 nm HR, ♦=560 nm HR;

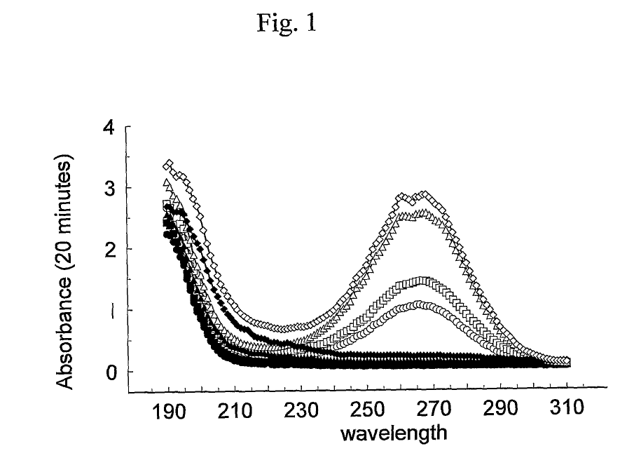

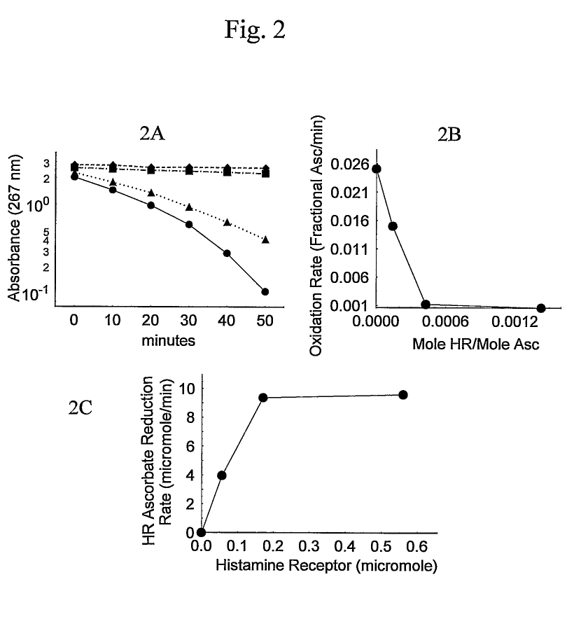

It should be noted that the plots set forth, e.g., in FIGS. 1 and 2 are intended to show the general characteristics of treatments and receptor effects among those of this invention, for the purpose of the description of such embodiments herein. These plots may not precisely reflect the characteristics of any given embodiment, and are not necessarily intended to define or limit specific embodiments within the scope of this invention.

### DETAILED DESCRIPTION

The following description is merely exemplary in nature and is not intended to limit the present disclosure, application, or uses.

The present application is related to U.S. Ser. No. 10/401,421 (filed Mar. 28, 2003) for Catecholamine Pharmaceutical Compositions and Methods; U.S. Ser. No. 60/672,224 (filed Apr. 15, 2005) for Ascorbate Binding Peptides; and U.S. Ser. No. 60/706,249 (filed Aug. 5, 2005) for GPCR Modulators; each of which is hereby incorporated by reference in its entirety.

The present invention encompasses certain novel compositions and methods for the administration of aminergic compounds to human or animal subjects. Specific compounds and compositions to be used in the invention must, accordingly, be utility-acceptable. As used herein, such a “pharmaceutically acceptable” component is one that is suitable for use with humans and/or animals without undue adverse side effects (such as toxicity, irritation, and allergic response) commensurate with a reasonable benefit/risk ratio.

The compositions and methods of this invention preferably comprise the administration of an aminergic compound and a complement compound, preferably at least levels in which one, e.g., molecule, of a complement compound can bind to a receptor to which one, e.g., molecule of the aminergic compound is bound. In one embodiment, the molar ratio between complement compound and aminergic can be about 1:1; in some embodiments, the molar ratio can range from about 1:10 or greater; in some embodiments, the molar ratio can range up to about 10:1.

Preferably, the levels can be synergistic in that the combination can provide an enhanced receptor-based effect, i.e. one that is greater than the sum of the effects exhibited when each of the complement alone and the aminergic alone is administered separately, i.e. the effect exhibited when an administered aminergic is without the presence of the complement and vice versa. Such effects include one or more of: increasing the degree of the effect of the aminergic compound on the receptor, such as intensifying a response or the inhibition of a response by the receptor; increasing the duration of the effect of the aminergic compound on the receptor; and making aminergic compounds effective on the receptor at dosage levels that would otherwise be ineffective. This includes enhancing the degree or duration of effect, or of permitting such equally effective lower doses, of aminergic receptor antagonists, as well as agonists. (As used herein, the word “include,” and its variants, is intended to be non-limiting, such that recitation of items in a list is not to the exclusion of other like items that may also be useful in the materials, compositions and methods of this invention. Also as used herein, the words “preferred” and “preferably” refer to embodiments of the invention that afford certain benefits, under certain circumstances. However, other embodiments can also be preferred, under the same or other circumstances. Furthermore, the recitation of one or more preferred embodiments does not imply that other embodiments are not useful and is not intended to exclude other embodiments from the scope of the invention.)

Thus, a composition according to the present invention can comprise at least one histaminergic, dopaminergic, muscarinergic, serotoninergic, octopaminergic, or trace aminergic compound, or at least one adrenergic antagonist compound; and can include no adrenergic agonist compound. Preferably, a histaminergic, dopaminergic, muscarinergic, serotoninergic, octopaminergic, or trace aminergic compound(s) selected for use in a composition according to the present invention can be a non-adrenergic histaminergic, dopaminergic, muscarinergic, serotoninergic, octopaminergic, or trace aminergic compound(s), i.e. one having no substantial modulatory effect on adrenergic receptors of the treated subject, preferably one having no significant effect thereon.

A composition according to the present invention can comprise a mixture of an aminergic compound and a complement compound, the mixture being any combination in which the aminergic compound is not stably attached to the complement compound at the molecular level, i.e. either they are physically separate or they can separate upon simple physical treatment such as dissolution in a solvent under biologically mild conditions (e.g., between pH 5.5 and pH 8.5, at a temperature below 50° C.). Thus, the mixture can have, e.g., a format comprising a juxtaposition of separate particles of each compound in any medium, or a solution or suspension of these compounds in any medium, a co-solidification (as by, e.g., melt-cooling or compression of these compounds solely with each other or jointly with at least one further component), a co-crystallization of the compounds with one another, or a salt formed between the aminergic and complement compounds.

### Aminergic Compounds

Aminergic compounds useful herein are functionally-acceptable compounds which directly or indirectly agonize or antagonize a biogenic amine receptor. In one embodiment, the aminergic compounds can be receptor binding site ligands, i.e. direct agonists or antagonists. A very large variety of aminergic compounds are known in the art; illustrative examples of aminergic compounds are provided below for major classes of: dopaminergic, histaminergic, muscarinergic, serotoninergic, octopaminergic, and trace aminergic compounds, as well as for adrenergic antagonist compounds. It is understood that aminergic compounds according to the present invention include pharmaceutically acceptable salts and esters thereof, and mixtures thereof, as well as precursors thereof that are capable of in vivo conversion thereto.

### Adrenergic Antagonist Compounds

Adrenergic compounds useful herein are functionally-acceptable compounds which directly or indirectly antagonize an alpha or beta-adrenoceptor, eliciting a sympathomimetic response. In one embodiment, the adrenergic antagonist compounds can be receptor binding site ligands, i.e. direct antagonists. Many adrenergic antagonist and other aminergic compounds are known in the art, including those described in Goodman and Gillnan's, The Pharmacological Basis of Therapeutics, 8^{th} Edition (1990) (incorporated by reference herein).

Adrenergic antagonist compounds useful herein include, e.g.: afluzosin, buflomedil, bunazosin, corynanthine, dapiprazole, dihydroergocornine, dihydroergocristine, dihydroergocryptine, dihydroergotamine, dihydroergotoxine, doxazosin, ergotamine, ergotoxine, guanadrel, guanethidine, idazoxan, ifenprodil, indoramin, mianserin, mirtazapine, moxisylyte, naftopidil, nicergoline, phenoxybenzamine, phentolamine, prazosin, raubasine, reserpine, tamsulosin, terazosin, thymoxamine, tolazoline, trimazosin, urapidil, and yohimbine (alpha-adrenergic antagonists); acebutolol, alprenolol, atenolol, befimolol, betaxolol, bevantolol, bisoprolol, bopindolol, bucindolol, bucumolol, bufetolol, bufuralol, bunitrolol, bupranolol, butidrine, butofilolol, capsinolol, carazolol, carteolol, carvedilol, celiprolol, cetamolol, cloranolol, dilevalol, diprafenone, epanolol, ersentilide, esmolol, esprolol, indenolol, landiolol, levobunolol, medroxalol, mepindolol, metipranolol, metoprolol, moprolol, nadolol, nadoxolol, nebivolol, nifenalol, oxprenolol, penbutolol, pindolol, practolol, pronethalol, propafenone, propranolol, sotalol, sulfinalol, talinolol, tertatolol, tilisolol, timolol, toliprolol, trimepranol, xamoterol, and xibenolol (beta-adrenergic antagonists); and amosulalol, arotinolol, labetalol, and nipradilol (alpha- and beta-adrenergic antagonists); derivatives thereof; precursors thereof; pharmaceutically acceptable salts and esters thereof; and combinations thereof.

In a composition according to an embodiment of the present invention an adrenergic agonist can be provided in a combination with a complement compound according to the present invention. Such adrenergic antagonists as are listed above are also useful therefor. In one embodiment of a method according to the present invention, any such adrenergic antagonist can be administered with a complement compound according to the present invention.

Adrenergic antagonist compounds are useful for treatment of a variety of disease and conditions. Alpha adrenergic antagonists are useful for treatment of, e.g.: vascular disorders, such as hypertension, migraine, vasospasicity (e.g., Raynauds disease-associated peripheral vasospasticity), vascular constriction (e.g., hyperconstriction) disorders, including vasoconstriction-associated necrosis (i.e. necrosis development at sites of high vasoconstrictor concentrations); nonvascular smooth muscle contraction (e.g., hypercontraction) disorders; neural or neurological disorders, such as sympathetic tone disorders, dyskinesias (e.g., in Parkinson's disease, tarditive dyskinesia), behavioral or mood disorders (e.g., depression, Alzheimer's disease-associated disruptive behaviors, such as irritability, uncooperativeness, aggression); hormone- or neurotransmitter-release or -secretion disorders, such as norepinephrine release inhibition, acetylcholine release inhibition, insulin secretion inhibition; lipolysis inhibition; genitourinary disorders, such as dysuria (e.g., urinary outlet obstruction), prostate disorders (e.g., benign prostatic hyperplasia, benign prostatic hypertrophy), sexual dysfunctions (e.g., erectile dysfunction, female sexual dysfunction); and pheochromocytoma.

Beta adrenergic antagonists are useful for treatment of, e.g.: cardiovascular disorders, such as hypertension, coronary artery disease (e.g., aortic aneurysm, development of Marfan's syndrome-associated aortic aneurysm), cardiac disorders (e.g., myocardial infarction, congestive heart failure, cardiomyopathy), angina, arrhythmia (e.g., recurrent ventricular tachycardia, recurrent ventricular fibrillation, pheochromocytoma-associated arrhythmia), hyperthyroidism-associated cardiovascular symptoms, and atrial fibrillation (e.g., for controlling atrial fibrillation rate); glaucoma; migraine; and panic disorder symptoms, e.g., tachycardia, muscle tremor.

### Dopaminergic Compounds

Dopaminergic compounds useful herein are functionally-acceptable compounds which directly or indirectly agonize or antagonize a dopamine receptor. In one embodiment, the dopaminergic compounds can be receptor binding site ligands, i.e. direct agonists or antagonists. Among the many dopaminergic compounds known in the art are, e.g., substituted dopamine derivatives, quinpirole, carmoxirole, 2-amino-5,6-dihydroxy-1,2,3,4-tetrahydronaphthalene, lisuride, pergolide, apomorphine, haloperidol, domperidone, metaclopramide, spiperone, haloperidol, diphenylbutylpiperidine, ecopipam, fenoldopam, fluphenazine, flupentixol, sulpiride, phenothiazines (e.g., thioridazine), thioxanthenes (e.g., chlorprothixene, flupenthixol, thiothixene, zuclopenthixol) naloxone, and bromocriptine; and derivatives thereof, precursors thereof, pharmaceutically acceptable salts and esters thereof, and combinations thereof. One example of a precursor to a dopaminergic compound is L-dopa (L-3,4-dihydroxyphenylalanine).

Dopaminergic compounds are useful in treatment of disorders including, e.g., neurological or neural disorders, such as Parkinson's disease (PD) (including, e.g., PD-associated akathesia, PD-associated musculoskeletal pain), psychiatric disorders, autism (e.g., infantile autism), ADHD, movement disorders (e.g., restless legs syndrome, periodic limb movement disorder, and pediatric movement disorders including, e.g., dopa-responsive dystonia); chemotherapy-induced nausea (emesis); hyperprolactinemia; heart failure; and obesity or obesity-indicative or -related conditions, such as hyperphagia, hyperglycemia, hyperlipidemia, and excess body fat storage.

### Histaminergic Compounds

Histaminergic compounds useful herein are functionally-acceptable compounds which directly or indirectly agonize or antagonize a histamine receptor. In one embodiment, the histaminergic compounds can be receptor binding site ligands, i.e. direct agonists or antagonists. Among the many histaminergic compounds known in the art are, e.g., substituted histamine derivatives, e.g., 4-methyl histamine, N-alpha-methylhistamine, R-alpha-methylhistamines, 2-phenylhistamines (e.g., 2-[3-(trifluoromethyl)phenyl]histamine, N-alpha-methyl-2-[3-(trifluoromethyl)phenyl]histamine); 2-(2-pyridyl)ethylamine, histaprodifen (2-[2-(3,3-diphenylpropyl)-1H-imidazol-4-yl]ethylamine), N-methyl-histaprodifen, N-alpha-2-[(1H-imidazol-4-yl)ethyl]histaprodifen; (6-[2-(4-imidazolyl)ethylamino]-N-(4-trifluoromethylphenyl) heptanecarboxamide); dexchlorpheniramine, diphenhydramine; amthamine, clozapine, clobenpropit, dimaprit, imetit, immepip, impromidine; (+)-chlorpheniramine, cimetidine, ciproxifan, clobenpropit, pyrilamine/mepyramine, ranitidine, terfenadine, thioperamide, tiotidine, and triprolidine; and derivatives thereof, precursors thereof, pharmaceutically acceptable salts and esters thereof, and combinations thereof.

Histaminergic compounds are useful in a variety of disorders including: treatment of, e.g., neurological disorders, such as attention-deficit hyperactivity disorders (ADHD), Alzheimer's disease, schizophrenia, cognitive dysfunctions, vigilance deficits, epilepsy, sleep-related disorders (e.g., sleep wakefulness disorders, hypersomnia, narcolepsy); optokinetic or vestibular disorders, such as motion sickness, vertigo; gastrointestinal disorders, such as gastric acid secretion disorders (e.g., gastroesophageal reflux disease or “acid reflux”), peptic ulcer, duodenal ulcer, gastric ulcer, stress ulcer; allergy, such as seasonal allergy (e.g., hay fever), asthma, urticaria, and symptoms thereof (e.g., rhinitis, conjunctivitis); nasopharyngeal infection, such as colds and influenzas; obesity; mood disorders, such as depression; and are used as sedatives and hypnotics.

### Muscarinergic Compounds

Muscarinergic compounds useful herein are functionally-acceptable compounds which directly or indirectly agonize or antagonize a muscarinic acetylcholine receptor. In one embodiment, the muscarinergic compounds can be receptor binding site ligands, i.e. direct agonists or antagonists. Among the many muscarinergic compounds known in the art are, e.g., substituted acetylcholine derivatives, aceclidine, arecoline, atropine, benzhexyl, benztropine, cevimeline, 2-ethyl-8-methyl-2,8-diazaspiro(4.5)decane-1,3-dione, R-(Z)-(+)-alpha-(methoxyimino)-1-azabicyclo[2.2.2]octane-3-acetonitrile, milameline, oxotremorine, pilocarpine, pirenzepine, scopolamine, talsaclidine, telenzepine, trihexyphenidyl, and xanomeline; and derivatives thereof, precursors thereof, pharmaceutically acceptable salts and esters thereof, and combinations thereof.

Muscarinergic compounds are useful in a variety of disorders including: treatment of, e.g., gastrointestinal disorders, such as gastrointestinal disease, gastric [hyper]secretion disorders (e.g., acid-peptic disorders, esophageal “acid” reflux disease), peptic ulcers, duodenal ulcers, gastroparesis, abdominal distension, gastric atony, irritable bowel syndrome; respiratory disorders, such as respiratory tract secretion disorders, bronchial dilation disorders (e.g., chronic bronchitis, emphysema, chronic obstructive pulmonary disease, i.e. COPD); urinary bladder disorders (e.g., urinary bladder contraction frequency in enurensis, voiding dysfunction in spastic paraplegia); cardiac disorders, such as (acute) myocardial infarction; neurological or neural disorders, such as parkinsonism, movement disorders (dystonias); optokinetic or vestibular disorders, such as motion sickness; oral disorders, such as a salivation disorder, e.g., excessive salivation, xerostomia; ocular disorders, such as glaucoma, iridocyclitis, or choroiditis (and as inducers of ocular mydriasis and cycloplegia); nasal disorders, such as rhinitis; and mushroom poisoning.

### Serotoninergic Compounds

Serotoninergic compounds useful herein are functionally-acceptable compounds which directly or indirectly agonize or antagonize a serotonin receptor. In one embodiment, the serotoninergic compounds can be receptor binding site ligands, i.e. direct agonists or antagonists. Among the many serotoninergic compounds known in the art are, e.g., substituted 5-hydroxy-tryptamine derivatives, e.g., 5-methoxytryptamine, a-methyl-5-hydroxytryptamine, 5-carboxamidotryptamine, 2-ethyl-5-methoxy-N,N-dimethyltryptamine; amphetamines, e.g., 2,5-dimethoxy-4-haloamphetamines, 2,5-dimethoxy-4-methamphetamine; ergotamine and lysergate derivatives, e.g., lysergic acid diethylamide, dihydroergotamine; almotriptan, buspirone, chlorpromazine, clozapine, cisapride, cyanopindolol, cyproheptadine, dexfenfluramine, dextromethorphan, dolasetron, donitriptan, eletriptan, eltoprazine, fenfluramine, fluoxetine, fluvoxamine, gepirone, granisetron, ketanserin, loxapine, meperidine, mesulergine, methiothepin, metergoline, methysergide, metoclopramide, mianserin, naratriptan, 1-naphthylpiperazine, nefazodone, olanzapine, ondansetron, paroxetine, pindolol, propranolol, risperidone, ritanserin, rizatriptan, spiperone, sertraline, sumatriptan, tropisetron, zolmitriptan, 8-hydroxy-dipropylaminotetralin, and 2-(2-methyl-4-chlorophenoxy)propanoic acid; and derivatives thereof, precursors thereof, pharmaceutically acceptable salts and esters thereof, and combinations thereof.

Serotoninergic compounds are useful in treatment of disorders including, e.g.: neurological and behavioral disorders, such as psychosis, schizophrenia, obsessive-compulsive disorder, autism (e.g., autism-associated aggressive, self-destructive behaviors, or knowledge deficits); headache, such as vascular headache (e.g., migraine, Horton's syndrome); mood disorders, such as anxiety, depression; gastrointestinal disorders, such as irritable bowel syndrome; and chemotherapy-induced nausea.

### Octopaminergic Compounds

Octopaminergic compounds useful herein are functionally-acceptable compounds which directly or indirectly agonize or antagonize an octopamine receptor. In one embodiment, the octopaminergic compounds can be receptor binding site ligands, i.e. direct agonists or antagonists. Octopaminergic compounds known in the art include, e.g.: agonists, such as octopamine, N-methyl octopamine; demethylchlordimeform; substituted phenyliminoimidazolidines (PR), such as NC 3 (trichloro PII), NC 5 (diethyl PII), NC 7 (2-methyl-4-chloro PII), and NC 13 (trimethyl PII; 2-(substituted phenylamino)-2-thiazolines, such as 2-(3-chlorophenylamino)-2-thiazoline; 2-(substituted phenylamino)-2-oxazolines, such as 2-(3-chlorophenylamino)-2-oxazoline; substituted phenylimidazolidines, such as 2-(2,6-diethylphenyl)imidazolidine and 1-(2,6-diethylphenyl)imidazolidine-2-thione; and others such as those reported in, e.g., A. Hirashima et al., Octopaminergic agonists for the cockroach neuronal octopamine receptor, J. Insect Sci., 3:10-18 (Apr. 21, 2003) (available online at http://insectscience.org/3.10); and antagonists, such as cyproheptadine, epinastine, mianserin, and phentolamine; and derivatives thereof, precursors thereof, pharmaceutically acceptable salts and esters thereof, and combinations thereof.

### Trace Aminergic Compounds

Trace aminergic compounds useful herein are functionally-acceptable compounds which directly or indirectly agonize or antagonize a trace amine receptor. In one embodiment, the trace aminergic compounds can be receptor binding site ligands, i.e. direct agonists or antagonists. Trace aminergic compounds known in the art include, e.g.: agonists, such as tryptamine; beta-phenylethylamine; tyramine, 3-methoxytyramine; 5-(4-hydroxyphenyl)oxazole; amphetamines, such as 3,4-methylenedioxymethamphetamine; ergotamines; normetanephrine, metanephrine; and antagonists, such as yohimbine; and derivatives thereof, precursors thereof, pharmaceutically acceptable salts and esters thereof, and combinations thereof.

The term “functionally acceptable” refers to the acceptability of a given method or material for a desired function, i.e. a desired purpose. This term is broader than, and encompasses, “pharmaceutically acceptable,” as well as other (e.g., non-pharmaceutically acceptable) classes of methods and materials. Examples of such other “functionally acceptable” classes include, but are not limited to:

biocidally acceptable (e.g., for animal/insect or human biocidal and/or toxicity-inducing purposes);

biostatically acceptable (e.g., for animal/insect or human juvenilization, infertility-producing, and/or contraceptive purposes);

deterrently acceptable (e.g., in regard to animal/insect or human repellent, irritant, pro-inflammatory, and/or pro-algesic purposes); and

calmatively or immobilizationally acceptable (e.g., in regard to non-medical purposes in which animal/insect or human central nervous system depression is desired, including those employing one or more of, e.g., sedative-hypnotic agents, anxiolytics, anesthetic agents, opioid analgesics, skeletal muscle relaxants, paralytic agents, and other agents capable of inducing sedation, relaxation, or immobilization).

Particular examples of such purposes include, e.g., criminal deterrence or immobilization, crowd control, wild animal and insect control (e.g., deterrence, repellence), and animal/insect population growth control. In some cases, materials or methods can be acceptable for multiple purposes; for example, a biostatically acceptable agent can also be pharmaceutically acceptable.

As used herein, the term “pharmaceutically acceptable” means suitable for use in, on, or with human and/or animal subjects or tissue(s) without undue adverse side effects (such as toxicity, irritation, and allergic response) commensurate with a reasonable benefit/risk ratio assessed with regard to the viability of the subject(s) and to other health factor(s) as may be considered important in sound medical judgment. In this, “pharmaceutical” refers to materials and methods that provide utility for any one or more of, e.g., prophylactic, curative, palliative, nutritive, cosmetic (e.g., biocosmetic, neurocosmetic), or diagnostic purposes, whether directly or indirectly. Examples illustrating indirect pharmaceutical utility include, but are not limited to, materials and methods employed as an adjunct to another treatment, e.g., an anesthetic or a muscle paralysis-inducing agent used in conjunction with a surgical treatment, or a detectable agent used to localize or visualize a mass to be targeted with radiation, or a label or tracer present in an administered formulation to permit verification of compliance with a treatment regimen. “Pharmaceutically acceptable” excipients (e.g., carriers and other additives) can further be materials that do not interfere with the effectiveness of the biological activity of the active ingredient(s) of a mixture, such as a pharmaceutical formulation, according to the present invention.

As used herein, the term subefficacious amount means an amount that is less than the minimal dose of an aminergic compound that would be needed, when administered alone, in order to provide a given GPCR-based effect. For example, a subefficacious amount of an antihistamine would be less than the minimal dose of that antihistamine needed, when administered alone (without a complement compound as described herein being administered with, or within about 1-2 hours of administration of, the antihistamine) to obtain a given antihistaminic effect.

### Aminergic Compound Complements

The compositions and methods of this invention comprise a compound which is a complement compound. As used herein, a “complement compound” is a compound (whether administered in the form of a salt, ester, or other form), that is capable of enhancing the effect of a direct or indirect, aminergic GPCR agonist or antagonist compound on its aminertic receptor. The enhancement can be an increased degree or duration of effect, or some other property of the aminergic compound-aminergic GPCR receptor interaction. In some embodiments, the “complement” is a compound which, in a given composition or method, binds to the aminergic compound used in said composition or method. Such “binding” is the formation of a complex through physical-chemical interaction of the complement with the aminergic compound, through means other than covalent bonding. Such binding is described in the following articles, incorporated by reference herein: Root-Bernstein and Dillon, “Molecular Complementarity I: The Complementarity Theory of the Origin and Evolution of Life.” J. Theoretical Biology 188: 447-449 (1997); and Root-Bernstein, “Catecholamines Bind to Enkephalins, Morphiceptin, and Morphine,” Brain Research Bulletin 18: 509-532 (1987).

Binding between a complement and an aminergic compound can be demonstrated through any physical, chemical, or immunological technique. Physicochemical methods include nuclear magnetic resonance imaging, ultraviolet or visible light spectroscopy, capillary or other forms of electrophoresis, high pressure liquid and other forms of chromatography, pH titration, and buffering. Chemical methods include procedures that can demonstrate binding such as affinity selection using gels, cellulose, glass, plastic, and/or other bound ligands. Immunological procedures that can demonstrate molecular complementarity include, double antibody diffusion (DAD), double antibody enzyme-linked immunosorption assay (DA-ELISA), in which antibody to the aminergic and antibody to its potential complements are prepared and tested to determine whether the pairs of antibodies bind to one another.

Preferred complements include those selected from the group consisting of an ascorbate, an opioid (such as morphine), a polycarboxylic acid chelator (such as EDTA), and derivatives thereof, analogs thereof, precursors thereof, pharmaceutically acceptable salts and esters thereof, and mixtures thereof. A “pharmaceutically acceptable salt” is a cationic salt formed at any acidic (e.g., carboxyl) group, or an anionic salt formed at any basic (e.g., amino) group. Many such salts are known in the art, as described in World Patent Publication 87/05297, Johnston et al., published Sep. 11, 1987 (incorporated by reference herein). Preferred cationic salts include the alkali metal salts (such as sodium and potassium), and alkaline earth metal salts (such as magnesium and calcium). Preferred anionic salts include the halides (such as chloride salts). A “pharmaceutically acceptable ester” is an ester that does not essentially interfere with the activity of the compounds used herein, or that is readily metabolized by a human or animal subject to yield an active compound.

Ascorbates include ascorbic acid and pharmaceutically derivatives and metabolites thereof. Preferred ascorbates include ascorbic acid, sodium ascorbate, calcium ascorbate, L-ascorbic acid, L-ascorbate, dehydrosoascorbic acid, dehydroascorbate, 2-methyl-ascorbic acid, 2-methyl-ascorbate, ascorbic acid 2-phosphate, ascorbic acid 2-sulfate, calcium L-ascorbate dihydrate, sodium L-ascorbate, ascorbylesters, and mixtures thereof. Ascorbic acid is a particularly preferred ascorbate. Polycarboxylic acid chelators include ethylendiamine tetraacetic acid (EDTA), diethylene triamine pentaacetic acid, pharmaceutically acceptable salts thereof, and mixtures thereof.

As referred to herein, an “opioid” is an opiate, synthetic opioid agonist, synthetic opioid partial agonist, a derivative thereof, pharmaceutically acceptable salt or ester thereof, or a mixture thereof. Preferred opioids include opiates and synthetic opioid agonists. Preferred opioids include alfentanil, apomorphine, benzomorphan, buprenorphine, butorphanol, codeine, dezocine, dihydrocodeine, dihydrocodeinone, diphenoxylate, endorphins (such as Met-enkephalin, Leu-enkephalin, dynorphin A, and dynorphin B), fentanyl, heroin (diacetylmorphine), hydrocodone, hydromorphone, kyotorphin, levorphanol, levomethadyl acetate, loperamide, malbuphine, meptazinol, methadone, meperidine, morphiceptin, morphine, nalbuphine, nalmefene, oxymorphone, oxycodone, pentazocine, propoxyphene, sufentanil, and mixtures thereof. Particularly preferred opioids are selected from the group consisting of alfentanil, apomorphine, benzomorphan, codeine, dihydrocodeine, dihydrocodeinone, diphenoxylate, endorphins (such as Met-enkephalin, Leu-enkephalin, dynorphin A, and dynorphin B), fentanyl, heroin (diacetylmorphine), hydrocodone, hydromorphone, kyotorphin, levorphanol, levomethadyl acetate, loperamide, malbuphine, methadone, meperidine, morphiceptin, morphine, nalmefene, oxymorphone, oxycodone, propoxyphene, sufentanil, and mixtures thereof; more preferably selected from the group consisting of apomorphine, morphiceptin, morphine, Leu-enkephalin, Met-enkephalin, and mixtures thereof.

Further complement compounds that can be used include those ascorbates, morphines, chelators; ascorbate analogs, morphine analogs, and EDTA analogs; and precursors thereof; described in U.S. Ser. No. 60/672,224 (filed Apr. 15, 2005) for Ascorbate Binding Peptides; and U.S. Ser. No. 60/706,249 (filed Aug. 5, 2005) for GPCR Modulators.

Other complement compounds that can be used, either separately or in addition to, the above-described complement compounds, include resveratrol family members and cysteine family members, including the analogs thereof.

As used herein, resveratrol family members are compounds having a structure of any of the: naturally occurring monomeric polyhydroxystilbenes; aglycosides of naturally glycosylated polyhydroxystilbenes; substituted derivatives of any of these, including C1-C4 alkyl-substituted derivatives, C5-C10 aryl- and cycloalkyl-substituted derivatives, and heteroatom-substituted (e.g., hydroxy-, sulfoxy-, halo-, amino-, C1-C4 alkylamino-, di(C1-C4 alkyl)amino-, C1-C4 alkoxy-, and C5-C10 aryloxy- and cycloalkoxy-) derivatives; dehydro homologs thereof; cis- and trans-isomers of any of the foregoing; the ethers (and thioethers), esters (and thioesters), amides, and salts of the foregoing, such as C1-C4 alkyl ethers, C5-C10 aryl and cycloalkyl ethers, glycosides, sulfoxy esters, nitroxy esters, phosphoxy esters, C1-C18 acyl and aroyl esters, and the salts thereof.

The substituted members of the resveratrol family can comprise one or more than one substituent. For example, a mono- or di-glycosylated resveratrol can be used. The sugar(s) in a glycosylated member of this family can be any mono-, di-, or oligo-saccharide of up to 5 monomers; thus, the sugar(s) or sugar monomers can be aldoses, ketoses, amino sugars, deoxy sugars, alkylene-bridged sugars, alkylidene-bridged sugars, sugar ethers (preferably methyl and/or ethyl ethers), sugar acids, sugar alcohols, and so forth. Piceid is one preferred example of a substituted member of the resveratrol family; in this case it is a glycosylated resveratrol, resveratrol-3-O-beta-glucoside.

Examples of naturally occurring monomeric polyhydroxystilbenes include: resveratrols; polydatins; gnetols; gnetucleistols; piceatannols; pinostilbenes; pterostilbenes; rhapontins; rhapontigenins; piceids; astringins; combretastatins; mulberrosides; and their dihydro-, oxy-, deoxy-, hydroxy-, methyl-, methoxy-derivatives, and isomeric variants. Viniferins and scirpusins are preferred examples of oligomeric (including dimeric) polyhydroxystilbenes. Resveratrol family members useful herein comprise at least two, preferably at least three, oxy groups (such as hydroxy, alkoxy, acyloxy, glycoxy, and so forth) attached to the phenyl groups of the stilbene, wherein each of the phenyl groups of the stilbene has at least one such oxy group as a substituent on the ring. In a preferred embodiment, at least one, and preferably at least two, of the oxy groups can be hydroxy groups.

In one embodiment, at least three hydroxy groups can be present in the polyhydroxystilbene. In one embodiment, the polyhydroxystilbene can comprise at least of the oxy groups at position 4′ of the stilbene, preferably a 4′-hydroxy group. In one embodiment, the polyhydroxystilbene can comprise oxy groups at positions 3, 5, and 4′ of the stilbene. In one embodiment, the polyhydroxystilbene can comprise a 3,5,4′-trihydroxystilbene. The polyhydroxystilbenes can be either cis- or trans-stilbenes. A mixture, such as racemic mixture can be used. In one embodiment, the polyhydroxystilbene(s) can be a polyhydroxy-trans-stilbene(s). In a preferred embodiment, the polyhydroxystilbene(s) can comprise a 4′-(oxy group)-trans-stilbene, preferably a 3,5,4′-tri(oxy group)-trans-stilbene; more preferably a 3,5,4′-trihydroxy-trans-stilbene.

In one preferred embodiment the resveratrol family member can have a structure of an isomer or native form of any of the: resveratrols; polydatins; gnetols; gnetucleistols; piceatannols; pinostilbenes; pterostilbenes; rhapontins; rhapontigenins; piceids; astringins; combretastatins; mulberrosides; or the dihydro-, oxy-, deoxy-, hydroxy-, methyl-, and/or methoxy-derivatives of the foregoing. A mixture can be used.

As used herein, cysteine family members are any of: cysteine, the N—(C1-C18 acyl)-cysteines (e.g., N-acetylcysteine), the N—(C1-C18 aroyl)-cysteines; cystine, the N—(C1-C18 acyl)-cystines (e.g., N-acetyl-cystine), the N—(C1-C18 aroyl)-cystines; taurine, the N—(C1-C18 acyl)-taurines (e.g., N-acetyl-taurine), the N—(C1-C18 aroyl)-taurines; and L-2-oxothiazolidine-4-carboxylic acid. These include members of any chirality or chiralities; a racemic mixture can be used. In one embodiment, the family member(s) used can be L-cysteine family member(s). In one preferred embodiment, the cysteine family member can be any of: cysteine, taurine, or the N—(C1-C18 acyl)-derivatives thereof, or mixtures thereof. Preferred N-acyl derivatives include the C1-C12, C1-C10, C1-C8, C1-C6, C1-C4, or C1-C2 N-acyl derivatives of cysteine and taurine.

Cysteine family members include monomeric compounds as described above, as well as homo-oligomers (including dimers) sand homo-polymers of any cysteine family member, and oligomers and polymers of cysteine family members with one another; yet, in the case of amino acyl polyamide oligomers and polymers, such polymers having amino acid residues that are not cysteine family member residue(s) can have amino acid sequence(s) that are not-naturally occurring amino acid sequences. As used herein, cysteine family members include those monomeric, oligomeric (including dimeric), and polymeric members that are derivatized, e.g., as by glycosylation, (non-aminoacyl-)amidation, esterification, and the like. In a preferred embodiment, a monomeric or dimeric cysteine family member can be used; preferably a monomeric member. A mixture can be used.

An aminergic compound can be covalently linked to a resveratrol family member or to a cysteine family member to provide a bifunctional compound. Such linking can be performed, e.g., as described in U.S. Ser. No. 60/706,249 (filed Aug. 5, 2005).

### Pharmaceutical Compositions

The compositions of this invention are preferably provided in unit dosage form. As used herein, a “unit dosage form” is a composition of this invention containing an amount of an aminergic compound and a complement compound that is suitable for administration to a human or animal subject, in a single dose, according to good medical practice.

### Aminergic Compound Dosage:

Compositions useful in the methods of this invention comprise a safe and effective amount of an aminergic compound and a safe and effective amount of a complement compound. In one embodiment, preferred compositions of this invention comprise a subefficacious amount of an aminergic compound. A “subefficacious amount” of a given aminergic compound is an amount which is safe and effective when administered to a human or animal subject in a composition or method of this invention, but which if administered without a complement compound would be less than the minimal amount needed to induce a given clinical effect. A “safe and effective” amount of an aminergic compound is an amount that is sufficient to have the desired therapeutic effect in the human or animal subject, without undue adverse side effects (such as toxicity, irritation, or allergic response), commensurate with a reasonable benefit/risk ratio when used in the manner of this invention. The specific safe and effective amount of the aminergic compound can vary with such factors as the particular condition being treated, the physical condition of the patient, the nature of concurrent therapy (if any), the specific aminergic compound used, the specific route of administration and dosage form, the carrier employed, and the desired dosage regimen.

In general, the amount of aminergic compound in a unit dose composition of this invention is preferably from about 1% to about 90%, preferably from about 10% to about 50%, of the uncomplemented clinically efficacious amount of said aminergic compound administered on a daily basis, divided by the number of doses of said compound to be given in a day. The “uncomplemented clinically efficacious amount” is that amount which is demonstrated to have a desired therapeutic effect according to good medical practice, without the administration of a complement compound. Preferably the uncomplemented clinically efficacious amount is that which is demonstrated in the art to have clinical utility in the treatment of the disorder to be treated, preferably through controlled clinical studies, more preferably as approved for commercial marketing. The “number of doses” for a given aminergic compound is the number of doses necessary to maintain an effective concentration of the compound at the site(s) at which the compound is to have a therapeutic effect. The uncomplemented clinically efficacious amount and number of doses can vary according to the aminergic compound and its pharmacokinetic characteristics, the disorder to be treated, and the route of administration. Preferably, the amount of aminergic compound in the compositions of this invention is equal to from about 1% to about 90%, preferably from about 10% to about 50%, of the amount of aminergic compound in the uncomplemented clinically efficacious compositions of the aminergic compound that are used in the art.

### Complement Compound Dosage:

The compositions of this invention also comprise a safe and effective amount of a complement compound. A “safe and effective amount” of a complement compound is an amount that is sufficient to increase the clinical efficacy of a given aminergic compound in a human or animal subject, without undue adverse side effects (such as toxicity, irritation, or allergic response), commensurate with a reasonable benefit/risk ratio when used in the manner of this invention. The specific “safe and effective amount” of the complement compound can vary with such factors as the particular aminergic compound used, the particular condition being treated, the physical condition of the patient, the duration of treatment, the nature of concurrent therapy (if any), the specific dosage form to be used, the carrier employed, the solubility of the compound therein, and the dosage regimen desired.

In some embodiments hereof, a composition can comprise: a safe and effective amount of a non-adrenergic aminergic, or an adrenergic antagonist, compound; and a hyperpreserving amount of an ascorbate, a hyperpreserving amount of a polycarboxylic acid chelator, a hyperpreserving amount of a resveratrol family member, or a mixture thereof. Other embodiments comprise: a subefficacious amount of an aminergic compound; and a complement compound, selected from the group consisting of hyperpreserving amounts any ascorbate, hyperpreserving amounts of any polycarboxylic acid chelator, hyperpreserving amounts of any resveratrol family member, and mixtures thereof.

As referred to herein, a “hyperpreserving amount” of an ascorbate, of a polycarboxylic acid chelator, or of a resveratrol family member, is an amount that is in excess of the amount conventionally used (the “preservative level”) to preserve an aminergic compound in a dosage form (e.g., to prevent the oxidation of an aminergic compound in solution). Preferably, the preservative level of the complement is that amount which is demonstrated to protect the aminergic compound in a clinical dosage form from degradation over a reasonable shelf life (e.g., two years) under typical storage conditions. Preferably the preservative level is that which is demonstrated in the art to have preservative utility in compositions comprising aminergic compounds, preferably at levels approved for commercial marketing of such products. In such embodiments of this invention, the dosage forms of this invention comprise a concentration of complement at least about 10, preferably at least about 25, preferably at least about 50, preferably at least about 100, preferably at least about 150, preferably about 200, times higher than the concentration of aminergic compound.

In a preferred embodiment, the preservative level is determined according to the following Antioxidant Effect Method. (As discussed above, other methods can be used, however.) In this method, a solution containing the aminergic compound (e.g., an antihistamine) is placed in a water-jacketed chamber maintained at 37° C. and the time noted. The solution is aerated with a gas mixture containing a known amount of oxygen and/or other gases. At different times, an aliquot of the solution is taken from the chamber and injected into a capillary electropherograph, which separates compounds based on their charge-to-mass ratio. (The conditions used for measuring aminergics are known in the art.) A sample is injected at a rate of 7.7 mL/sec for 2 seconds into a 98 cm capillary using vacuum injection. The sample is subjected to a 20 kV-to-ground driving force. The carrier buffer is 25 mM sodium borate at pH 9.4. Peaks representing an aminergic appear at pre-determinable times at a detection window in the capillary and are measured by the change in absorbance at 195 μm. Oxidation produces a different charge-to-mass ratio in the aminergic(s), and the oxidized compounds appear at a different time than the unoxidixed forms. For example, oxidized terfenadine (e.g., as 4-(hydroxydiphenylmethyl)-piperidine; and/or as a terfenadine alcohol or carboxylate metabolite)) appears as an earlier peak or peaks than that of unoxidized terfenadine. The size of the unoxidized peak is measured. The logarithm of the fraction of the oxidized peak remaining is plotted against the time since the solution was first placed in the chamber. From this plot, a slope is calculated. The equation determined in this way is: F=1−e^{-t/τ}; where “F” is the fraction oxidized, “e” is the natural logarithm, ‘t’ is the time since the solution is placed in the chamber, and “τ” is the exponential time constant, where, when t=τ, F=63.2% oxidized. The time constant is an inverse measure of the oxidation rate, where an increase in the time constant indicates a decrease in the rate of oxidation.

To determine the effect and preservative level of a complement (e.g., ascorbic acid, “AA,” or other ascorbate) on an aminergic compound (e.g., a terfenadine), the complement compound is placed in the solution with the aminergic compound. The solutions are treated as described above. There is a different anti-oxidant concentration in each solution, but a constant concentration ofterfenadine. The oxidation rates are measured in the manner described above and a different value of τ, the oxidation rate of the terfenadine, is determined for each concentration of anti-oxidant. There is a sigmoidal relation between the oxidation rate in the absence of the anti-oxidant (τ₀) and rates with increasing anti-oxidant concentration. The asymptote (τₘₐₓ) approached as the concentration of anti-oxidant increases is determined using a linear least-squares fit of the log of the anti-oxidant concentration plotted against the log of the ratio of the (τₘₐₓ/τ₀), with an iterative value of τₘₐₓ used until the error is minimized. The half-maximal inhibition of the terfenadine oxidation occurs when (τₘₐₓ−τ)=(τ−τ₀). The antioxidant concentration at the half-maximal inhibition ratio is the ratio of the amount of complement to the terfenadine that reduces the rate of terfenadine oxidation by one-half of the maximal reduction in terfenadine oxidation. In a preferred embodiment of this invention, the dosage forms of this invention comprise a concentration of complement at least about 2, preferably at least about 10, preferably at least about 25, preferably at least about 50, preferably at least about 100, preferably at least about 150 times, times higher than the preservative level of complement compound as determined by the at the half-maximal inhibition ratio as determined by the Antioxidant Effect Method. Preferably, the concentration of complement is at least about 2, at least about 10, preferably at least about 25, preferably at least about 50, preferably at least about 100, preferably at least about 150 times higher, preferably at least about 200, times higher than the antioxidant concentration at the half-maximal inhibition ratio determined by the Antioxidant Effect Method.

In some preferred embodiments, the amount of ascorbate, opiate, polycarboxylate chelator, resveratrol family member, cysteine family member, or analog compounds used as the complement compound(s) is preferably from about 10 micromolar to 10 millimolar, more preferably from about 100 micromolar to 1 about millimolar for aqueous solutions and suspensions. In some embodiments, the composition comprises from about 1 mg to about 1 gram of complement per milligram of aminergic, more preferably from about 10 mg to about 100 mg complement per milligram of aminergic. In some embodiments, for oral dosage forms, the compositions of this invention deliver from about 500 mg and 5 grams of ascorbate per day (or a molar equivalent of an ascorbate analog). For compositions comprising an opioid (or analog), low levels of opioid are preferably used to avoid systemic effects. In such opioid-containing compositions, preferably the composition can deliver from about 0.01 mg/70 kg of body weight to about 1 mg/70 kg of body weight per day in solutions of from about 0.01 to about 1 mg/mL solutions or suspensions, or in pills, inhalant, or other solid forms comprised of less than 1 mg/daily dose (or a molar equivalent for an analog). Preferably the levels of opioid are subefficacious. A “subefficacious amount” of an opioid is an amount which is safe when administered to a human or animal subject in a composition or method of this invention, but does not create a clinically significant narcotic effect. For compositions comprising a polycarboxylic acid chelator (or analog), compositions preferably comprise solutions of from about 1 micromolar to about 100 micromolar concentration, more preferably from about 5 to about 20 micromolar concentrations (or a molar equivalent for an analog). Such compositions are administered at no more than a total of 1.5 mg/dose or 1.5 mg/minute (during infusion or i.v. drip, etc.) and preferably at less than 0.15 mg/dose or 0.15 mg/minute.

For compositions comprising a resveratrol family member (or analog), compositions preferably comprise solutions of from about 1 micromolar to about 100 micromolar concentration, more preferably from about 5 to about 20 micromolar concentrations (or a molar equivalent for an analog). Such compositions are administered at no more than a total of 1.5 mg/dose or 1.5 mg/minute (during infusion or i.v. drip, etc.) and preferably at less than 0.15 mg/dose or 0.15 mg/minute.

For compositions comprising a cysteine family member (or analog), compositions preferably comprise solutions of from about 1 micromolar to about 100 micromolar concentration, more preferably from about 5 to about 20 micromolar concentrations (or a molar equivalent for an analog). Such compositions are administered at no more than a total of 1.5 mg/dose or 1.5 mg/minute (during infusion or i.v. drip, etc.) and preferably at less than 0.15 mg/dose or 0.15 mg/minute.

### Dosage Forms and Optional Materials:

The compositions of this invention can be in any of a variety of forms, suitable (for example) for oral, rectal, topical or parenteral administration. Depending upon the particular route of administration desired, a variety of pharmaceutically-acceptable carriers well-known in the art can be used. These include solid or liquid fillers, diluents, hydrotropes, surface-active agents, and encapsulating substances. Optional pharmaceutically-active materials can be included, which do not substantially interfere with the activity of the aminergic compounds. The amount of carrier employed in conjunction with the aminergic and complement compounds is sufficient to provide a practical quantity of material for administration per unit dose. Techniques and compositions for making dosage forms useful in the methods of this invention are described in the following references, all incorporated by reference herein: 7 Modem Pharmaceutics, Chapters 9 and 10 (Banker & Rhodes, editors, 1979); Lieberman et al., Pharmaceutical Dosage Forms: Tablets (1981); and Ansel, Introduction to Pharmaceutical Dosage Forms 2d Edition (1976); and U.S. Pat. No. 5,646,139, White et al., issued Jul. 8, 1997.

In particular, pharmaceutically-acceptable carriers for systemic administration include sugars, starches, cellulose and its derivatives, malt, gelatin, talc, calcium sulfate, vegetable oils, synthetic oils, polyols, alginic acid, phosphate buffer solutions, emulsifiers, isotonic saline, and pyrogen-free water. Preferred carriers for parenteral administration include propylene glycol, ethyl oleate, pyrrolidone, ethanol, and sesame oil. Preferably, the pharmaceutically-acceptable carrier, in compositions for parenteral administration, comprises at least about 90% by weight by the total composition.

Various oral dosage forms can be used, including such solid forms as tablets, capsules, granules and bulk powders. Tablets can be compressed, tablet triturates, enteric-coated, sugar-coated, film-coated, or multiple-compressed, containing suitable binders, lubricants, diluents, disintegrating agents, coloring agents, flavoring agents, flow-inducing agents, and melting agents. Liquid oral dosage forms include aqueous solutions, emulsions, suspensions, solutions and/or suspensions reconstituted from non-effervescent granules, and effervescent preparations reconstituted from effervescent granules, containing suitable solvents, preservatives, emulsifying agents, suspending agents, diluents, sweeteners, melting agents, coloring agents and flavoring agents. Preferred carriers for oral administration include gelatin, propylene glycol, cottonseed oil and sesame oil.

The compositions of this invention can also be administered topically to a subject, i.e., by the direct laying on or spreading of the composition on the epidermal or epithelial tissue of the subject. Such compositions include, for example, lotions, creams, solutions, gels and solids, and can, for example, be locally or systemically administered transdermally or by intranasal, pulmonary (e.g., by intrabronchial inhalation), ocular, or other mucosal delivery. Suitable carriers for topical administration on skin preferably remain in place on the skin as a continuous film, and resist being removed by perspiration or immersion in water. Generally, the carrier is organic in nature and capable of having dispersed or dissolved therein the aminergic and complement compounds. The carrier can include pharmaceutically-acceptable emollients, emulsifiers, thickening agents, and solvents.

Formulations suitable for mucosal administration by inhalation include compositions of the aminergic and complement compounds in a form that can be dispensed by inhalation devices among those known in the art. Such formulations preferably comprise liquid or powdered compositions suitable for nebulization and intrabronchial use, or aerosol compositions administered via an aerosol unit dispensing metered doses. Suitable liquid compositions comprise the active ingredient in an aqueous, pharmaceutically acceptable inhalant solvent, e.g., isotonic saline or bacteriostatic water. The solutions are administered by means of a pump or squeeze-actuated nebulized spray dispenser, or by any other conventional means for causing or enabling the requisite dosage amount of the liquid composition to be inhaled into the lungs.

Suitable powder compositions include, by way of illustration, powdered preparations of the active ingredients thoroughly intermixed with lactose or other inert powders acceptable for intrabronchial administration. The powder compositions can be administered via an aerosol dispenser or encased in a breakable capsule which can be inserted by the patient into a device that punctures the capsule and blows the powder out in a steady stream suitable for inhalation. Aerosol formulations preferably include propellants, surfactants and co-solvents and can be filled into conventional aerosol containers that are closed by a suitable metering valve.

### Methods of Treatment

This invention also provides methods of treating disorders associated with the regulation of an aminergic receptor. Methods of this invention include those comprising:
(a) administering a low dose of an aminergic compound, and
(b) administering a safe and effective of a complement compound.
   Other methods of this invention comprise:
(a) administering to a subject a safe and effective amount of an aminergic compound; and
(b) administering to a subject a complement compound, selected from the group consisting of a hyperpreserving amount of an ascorbate, a safe and effective amount of an opioid, a hyperpreserving amount of a polycarboxylic acid chelator, a hyperpreserving amount of a resveratrol family member, a hyperpreserving amount of a cysteine family member, and mixtures thereof.

The aminergic compound and the complement compound can be administered concomitantly, or separately. Preferably the aminergic and complement compounds are administered in a dosage regimen that results in efficacious levels of the compounds in the tissues that are to be treated throughout the desired duration of treatment. Preferably the aminergic and complement compounds are administered within one hour of each other, more preferably within ten minutes, more preferably at the same time. In some embodiments, a combination of the aminergic and complement is administered. Thus, methods involving administering an aminergic and administering a complement thereto include: one-step methods for administering the two types of compounds concomitantly, i.e. in combination form; two-step methods for separately administering the two types of compounds at different times; and two-step methods for concomitantly administering the two types of compounds, i.e. independently of one another.

The aminergic compounds and complements of this invention can be administered topically or systemically. Systemic application includes any method of introducing the compounds into the tissues of the body, e.g. intrathecal, epidural, caudal, intramuscular, transdermal, intra-arterial, intra-cardiac, intravenous, intraperitoneal, subcutaneous, sublingual, rectal, nasal, pulmonary, and oral administration. The specific dosage of compounds to be administered, as well as the duration of treatment, are mutually dependent. The dosage and treatment regimen can also depend upon such factors as the specific compound used, the ability of the compound to reach therapeutic concentrations at the site of the action, the nature and extent of other disorders (if any), the personal attributes of the subject (such as weight), compliance with the treatment regimen, the nature of concomitant therapies (if any), and the presence and severity of any side effects of the treatment.

A “low dose” of a given aminergic compound is from 1% to about 90%, preferably from about 10% to about 50%, of the uncomplemented clinically effective dose of said aminergic compound that would be administered to a human or animal subject over a given period of time to obtain a given level of effect. The methods of this invention can be effected by the administration of the aminergic compound at levels lower than practiced in the art, by administering the aminergic compound at dosage frequencies longer than practiced in the art, or both. The methods of this invention preferably use less drug to get the same (or greater) effect over the same (or greater) period of time; provide a greater effect using the same (or less) amount of drug over the same (or less) period of time; or afford longer duration of efficacy at the same (or greater) effect using the same (or less) amount of drug. Accordingly, the methods of this invention include methods wherein the compositions of this invention are administered in a number of doses equivalent to the number of doses of an aminergic compound used in the art, but a reduced dosage levels. Methods of this invention also include methods wherein compositions in the art are administered at the same unit dosage amount, but with reduced frequency. Preferably, the total amount of aminergic compound administered according to this invention during a given period of time is equal to from about 1% to about 90%, preferably from about 10% to about 50%, of the product of the number of doses of the drug administered in the art, multiplied by the amount of aminergic compound administered in each dose in the art, during the given period of time.

The methods of this invention involve administration of an aminergic compound and a complement compound to a human or animal subject for the treatment or prevention of any disorder which is mediated by an alpha- or beta-receptor. Such methods include, without limitation, those which have an affect on blood pressure, the vascular system, the heart, smooth muscles, or metabolism. Such neurological disorders include schizophrenia, Parkinson's disease and attention-deficit hyperactivity disorder. Cardiac disorders include hypotension, forward failure, backward failure and congestive heart failure. Vascular disorders include shock, hypotension, hemorrhage, and disorders associated with anesthesia. Respiratory disorders include nasal congestion, oral and nasal inflammation and swelling (such as caused by cold or flu), chronic obstructive pulmonary disease, asthma, emphysema, and bronchospasm. Gastrointestinal disorders include colic and Crohn's disease. Other disorders and uses include anaphylaxis, interstitial cystitis, overactive bladder syndrome, premature labor, myethsenia gravis, glaucoma, dilation of pupils, and weight reduction.

The compositions and methods of this invention also include the administration of an aminergic compound to cause homeostasis for topical anesthetics, increasing the duration of anesthetic action. Such anesthetics are administered, for example, by intramuscular injection during dental procedures or skin surgery. Accordingly, such compositions and methods of this invention additionally comprise a safe and effective amount of an anesthetic agent such as lidocaine or procaine. In a preferred embodiment, the level of the anesthetic agent is administered in a lower dose, with less volume of material injected, yielding an equivalent level and duration of anesthesia as conventional compositions that do not contain a complement.

The present invention also provides methods of determining a regimen for regulating an aminergic receptor in human or animal subjects, comprising:
(a) selecting a non-adrenergic aminergic or a an adrenergic antagonist (preferably a non-adrenergic aminergic) compound useful for regulating said receptor;
(b) selecting a complement compound;
(c) determining the dosage level and frequency of dosing of said aminergic compound for use in regulating said receptor when administered to said subjects in the absence of said complement;
(d) evaluating the effectiveness of said aminergic compound in regulating said receptor when administered to said subjects in the presence of said complement, as a function of the dosage level of said aminergic compound and the dosage level of said complement; and
(e) determining a regimen for regulating said receptor in said subjects by
   (i) selecting a dose level of said aminergic compound which is determined to be effective in said evaluating step (d) and that is lower than the dosage level determined in said step (c);
   (ii) selecting a dosage frequency that is determined to be effective in said evaluating step (d) and is longer than the dosage frequency determined in said step (c), or
   (iii) both (i) and (ii).
      In some embodiments, the selecting step (b) can comprise identifying the complement using any of the physical, chemical or immological techniques described above regarding complement binding. Preferably, step (c) for determining the dosage level and frequency of dosing in the absence of the complement is performed as discussed above regarding the uncomplemented clinically efficacious amount of aminergic compound. As used herein, the “absence” of the complement refers to levels of complement at the site of action of the aminergic compound that are not significant, preferably no higher than those associated with typical dietary levels of such complements. Preferably, step (d) for evaluating the effectiveness in the presence of said complement is performed as discussed above regarding the subefficacious levels aminergic compound in the dosage forms of this invention. As used herein, the “presence” of the complement refers to concurrent presence of the aminergic compound and the complement at the site of action of the aminergic compound.

The following non-limiting examples illustrate the compositions and methods of the present invention.

### Example 1

The availability of human H1 histamine receptor (Jena Bioscience, Jena, Germany) allows direct experiments of the effect of Asc on the receptor. 500 μL of commercial H1 histamine receptor (HR) is washed with 20 mM sodium phosphate buffer, pH 7.4, with sonication, centrifugation, and resuspension. HR is thus present as a 14.3 μM HR suspension in sodium phosphate buffer, pH 7.4. Ascorbate is prepared as a 10 mM stock in 20 mM sodium phosphate buffer, pH 7.4.

Triplicate samples of 255 μL containing 392±1.7 μM Asc (94.1 μg/ml) and 0.56 μM (31.4 μg/ml), 0.17 μM (9.4 μg/ml), or 56 nM (3.1 μg/ml) HR receptor are prepared. H1 histamine receptor has a molecular weight of 55.8 kDal. 200 μl of each sample are added to a 96 well plate and spectra collected from 190-310 nm in 1 nm increments during a series of repeated readings over a period of one hour in a Spectramax spectrophotometer. Difference spectra (versus absorbance readings for Asc or HR alone) have been used previously to quantitatively study binding of ascorbate. This process allows measurement of the effect of the presence of HR on ascorbate oxidation. The effect of HR on the ascorbate oxidation rate can be measured by the disappearance of the ascorbate resonance, with a peak at 267 nm. The ascorbate oxidation rates are measured by plotting the logarithm of the ascorbate versus time, with the rate constant determined from the calculated slope. Experiments demonstrate that HR concentrations in excess of 170 nM virtually stop ascorbate oxidation. The reduction of oxidized ascorbate by HR is calculated as the difference between the oxidation rate for ascorbate alone and the oxidation rate of ascorbate in the presence of HR. The calculation of mole Asc reduction/mole HR is made using the lowest HR concentration, 56 nM, well below the HR concentration at which this concentration of Asc reduction saturates. Results are presented in FIGS. 1 and 2.

FIG. 1 presents spectrograms demonstrating binding of ascorbate to the human H1 histamine receptor (HR) in in vitro suspensions. In this Figure, the closed symbols are the spectra of different concentrations of HR receptor without ascorbate; and the open symbols are spectra of HR plus 392 μM initial ascorbate; both are measured at 20 minutes after addition of ascorbate. The large peak centered at 267 nm represents unoxidized ascorbate. As ascorbate oxidizes, its absorbance disappears. However, these data indicate that HR helps maintain ascorbate in its anion form, e.g., possibly by reducing oxidized ascorbate. Subtraction of control spectrograms generated for ascorbate in buffer without HR, which represents background levels of ascorbate oxidation, reduces the absorbance reading in the 190-205 nm range, but leaves significant peaks centered at 267 nm for all three of the samples containing HR (data not shown, but compare the upper three spectrograms with the fourth highest spectrogram, which represents Asc with no added HR).

Data for the effect of H1 histamine receptor (HR) on ascorbate oxidation rates are shown in FIG. 2. FIG. 2A represents the ascorbate absorbance at 267 nm, i.e. the peak height of the highest spectral peak, 267 nm being the strongest Asc absorbance wavelength. The data are measured over time for various HR concentrations as absorbance difference spectra. The steepest declining curve shows the oxidation of ascorbate in the absence of HR. The upper two curves are virtually flat, indicating that there is little or no net oxidation of ascorbate in the presence of this amount of HR. From the plotted data, the rate constants were calculated. Experimental results show that 1.7 μM HR prevents ascorbate oxidation similar to that of 560 nM HR (data not shown). In FIG. 2B, the calculated rate constants for ascorbate oxidation at different HR concentrations are plotted as a function of the ratio of moles of HR to moles of ascorbate. Above a ratio of 0.0004 mole HR/mole ascorbate, there is no significant net oxidation of ascorbate during this time period. The prevention of oxidation at such low ratios indicates that the oxidation protection is not solely due to direct HR-ascorbate binding. FIG. 2C shows the rate of HR reduction of oxidized ascorbate, calculated as the difference between the oxidation rate of ascorbate alone and the rate in the presence of a given concentration of HR. The initial slope indicates the highest rate of HR reduction of ascorbate measured in these experiments. This value was calculated as 71 μmoles/min of Asc reduction per μmole of HR.

1-277. (canceled)

278. The pharmaceutical composition according to claim 277, wherein said complement is EDTA.

279. The pharmaceutical composition according to claim 274, wherein said complement comprises an opioid.

280. The pharmaceutical composition according to claim 279, wherein said opioid is selected from the group consisting of alfentanil, apomorphine, benzomorphan, buprenorphine, butorphanol, codeine, dezocine, dihydrocodeine, dihydrocodeinone, diphenoxylate, Met-enkephalin, Leu-enkephalin, dynorphin A, dynorphin B, fentanyl, heroin, hydrocodone, hydromorphone, kyotorphin, levorphanol, levomethadyl acetate, loperamide, malbuphine, meptazinol, methadone, meperidine, morphiceptin, morphine, nalbuphine, nalmefene, oxymorphone, oxycodone, pentazocine, propoxyphene, sufentanil, and mixtures thereof.

281. The pharmaceutical composition according to claim 274, wherein said complement comprises a resveratrol family member.

282. The pharmaceutical composition according to claim 281, wherein said resveratrol family member is selected from the group consisting of resveratrols, polydatins, gnetols, gnetucleistols, piceatannols, pinostilbenes, pterostilbenes, rhapontins, rhapontigenins, piceids, astringins, combretastatins, mulberrosides, and mixtures thereof.

283. The pharmaceutical composition according to claim 274, wherein said complement comprises a cysteine family member.

284. The pharmaceutical composition according to claim 283, wherein said cysteine family member is selected from the group consisting of cysteine, taurine, N—(C1-C18 acyl)-cysteine, N—(C1-C18 acyl)-taurine, and mixtures thereof.

285. The pharmaceutical composition according to claim 274, wherein said composition is suitable for oral administration.

286. The pharmaceutical composition according to claim 274, wherein said composition is suitable for topical administration.

287. The pharmaceutical composition according to claim 286, wherein said complement is an ascorbate.

288. The pharmaceutical composition according to claim 287, wherein said ascorbate is present at a level of from about 0.01 millimolar to about 5 millimolar concentration.

289. The pharmaceutical composition according to claim 286, wherein said administration is transdermal.

290. The pharmaceutical composition according to claim 286, wherein said administration is intranasal or pulmonary.

291. The pharmaceutical composition according to claim 286, wherein said administration is ocular.

292. The composition according to claim 273, wherein said non-adrenergic aminergic compound is a dopaminergic compound selected from the group consisting of quinpirole, carmoxirole, 2-amino-5,6-dihydroxy-1,2,3,4-tetrahydronaphthalene, lisuride, pergolide, apomorphine, haloperidol, domperidone, metaclopramide, spiperone, haloperidol, diphenylbutylpiperidine, ecopipam, fenoldopam, fluphenazine, flupentixol, sulpiride, phenothiazines, thioxanthenes, naloxone, bromocriptine, substituted dopamine derivatives; pharmaceutically acceptable salts or esters thereof; and combinations thereof.

293. The composition according to claim 273, wherein said non-adrenergic aminergic compound is a histaminergic compound selected from the group consisting of 2-(2-pyridyl)ethylamine, histaprodifen (2-[2-(3,3-diphenylpropyl)-1H-imidazol-4-yl]ethylamine), N-methyl-histaprodifen, N-alpha-2-[(1H-imidazol-4-yl)ethyl]histaprodifen, (6-[2-(4-imidazolyl)ethylamino]-N-(4-trifluoromethylphenyl)heptanecarboxamide), dexchlorpheniramine, diphenhydramine; amthamine, clozapine, clobenpropit, dimaprit, imetit, immepip, impromidine, (+)-chlorpheniramine, cimetidine, ciproxifan, clobenpropit, pyrilamine, mepyramine, ranitidine, terfenadine, thioperamide, tiotidine, triprolidine, substituted histamine derivatives; pharmaceutically acceptable salts or esters thereof; and combinations thereof.

294. The composition according to claim 273, wherein said non-adrenergic aminergic compound is a muscarinergic compound, selected from the group consisting of aceclidine, arecoline, atropine, benzhexyl, benztropine, cevimeline, 2-ethyl-8-methyl-2,8-diazaspiro(4.5)decane-1,3-dione, R-(Z)-(+)-alpha-(methoxyimino)-1-azabicyclo[2.2.2]octane-3-acetonitrile, milameline, oxotremorine, pilocarpine, pirenzepine, scopolamine, talsaclidine, telenzepine, trihexyphenidyl, xanomeline, substituted acetylcholine derivatives; pharmaceutically acceptable salts or esters thereof; and combinations thereof.

295. The method according to claim 273, wherein said non-adrenergic aminergic compound is a serotoninergic compound, selected from the group consisting of amphetamines, ergotamine, lysergate derivatives, almotriptan, buspirone, chlorpromazine, clozapine, cisapride, cyanopindolol, cyproheptadine, dexfenfluramine, dextromethorphan, dolasetron, donitriptan, eletriptan, eltoprazine, fenfluramine, fluoxetine, fluvoxamine, gepirone, granisetron, ketanserin, loxapine, meperidine, mesulergine, methiothepin, metergoline, methysergide, metoclopramide, mianserin, naratriptan, 1-naphthylpiperazine, nefazodone, olanzapine, ondansetron, paroxetine, pindolol, propranolol, risperidone, ritanserin, rizatriptan, spiperone, sertraline, sumatriptan, tropisetron, zolmitriptan, 8-hydroxy-dipropylaminotetralin, 2-(2-methyl-4-chlorophenoxy)propanoic acid, substituted 5-hydroxy-tryptamine derivatives; pharmaceutically acceptable salts or esters thereof; and combinations thereof.

296. A pharmaceutical composition comprising:
(a) a subefficacious amount of an aminergic compound; and
(b) a safe and effective amount of a complement compound that is any of the resveratrol family members or cysteine family members.

297. A pharmaceutical composition comprising:
(a) a safe and effective amount of a non-adrenergic aminergic compound; and
(b) a complement compound selected from the group consisting of hyperpreserving amounts of ascorbates, analogs and substituted derivatives thereof; safe and effective amounts of morphines, analogs and substituted derivatives thereof; hyperpreserving amounts of polycarboxylic acid chelators, analogs and substituted derivatives thereof; hyperpreserving amounts of resveratrol family members, analogs and substituted derivatives thereof; safe and effective amounts of cysteine family members, analogs and substituted derivatives thereof; and mixtures thereof.

298. The composition according to claim 297, wherein said non-adrenergic aminergic compound comprises a histaminergic, dopaminergic, muscarinergic, serotoninergic, octopaminergic, or trace aminergic compound.

299. The pharmaceutical composition according to claim 297, wherein said complement comprises an ascorbate.

300. A method for treating a disorder associated with a non-adrenergic biogenic amine receptor in a human or animal subject, comprising:
(a) administering to said subject a low dose of a non-adrenergic aminergic compound; and
(b) administering to said subject a safe and effective amount of a complement compound.

301. The method according to claim 300, wherein said non-adrenergic aminergic compound comprises a histaminergic, dopaminergic, muscarinergic, serotoninergic, octopaminergic, or trace aminergic compound.

302. The method according to claim 300, wherein said complement compound is selected from the group consisting of ascorbates, opioids, polycarboxylic acid chelators, resveratrol family members, cysteine family members, analogs and substituted derivatives thereof, and mixtures thereof.

303. The method according to claim 302, wherein said complement comprises an ascorbate.

304. The method according to claim 300, wherein:
(a) said aminergic compound is a histaminergic, dopaminergic, muscarinergic, or serotoninergic compound; and
(b) said complement is selected from the group consisting of hyperpreserving amounts of ascorbates, analogs and substituted derivatives thereof; safe and effective amounts of morphines, analogs and substituted derivatives thereof; hyperpreserving amounts of polycarboxylic acid chelators, analogs and substituted derivatives thereof; hyperpreserving amounts of resveratrol family members, analogs and substituted derivatives thereof; safe and effective amounts of cysteine family members, analogs and substituted derivatives thereof; and mixtures thereof.

305. The method according to claim 301, wherein non-adrenergic aminergic compound is a dopaminergic compound, and said method is a method for the treatment of a neurological or neural disorder, heart failure, hyperprolactinemia, obesity or an obesity-indicative or -related condition.

306. The method according to claim 301, wherein said non-adrenergic aminergic compound is a histaminergic, and said method is a method for the treatment of a neurological, mood, optokinetic, vestibular, or gastrointestinal disorder or the treatment of allergy, nasopharyngeal infection, or obesity.

307. The method according to claim 301, wherein said non-adrenergic aminergic compound is a muscarinergic compound, and said method is a method for the treatment of a gastrointestinal, respiratory, neurological, neural, nasal, oral, ocular, urinary bladder, or cardiac disorder, or the treatment of motion sickness.

308. The method according to claim 301, wherein said non-adrenergic aminergic compound is a serotoninergic compound, and said method is a method for the treatment of a neurological, behavioral, or gastrointestinal disorder, or the treatment of headache.

309. A method for treating a disorder associated with a non-adrenergic biogenic amine receptor in a human or animal subject, comprising:
(a) administering to said subject a safe and effective amount of a non-adrenergic aminergic compound; and
(b) administering to said subject a complement compound selected from the group consisting of hyperpreserving amounts of ascorbates, analogs and substituted derivatives thereof; safe and effective amounts of opiates, analogs and substituted derivatives thereof; hyperpreserving amounts of polycarboxylic acid chelators, analogs and substituted derivatives thereof; hyperpreserving amounts of resveratrol family members, analogs and substituted derivatives thereof; safe and effective amounts of cysteine family members, analogs and substituted derivatives thereof; and mixtures thereof.

310. The method according to claim 309, wherein said non-adrenergic aminergic compound is a histaminergic, dopaminergic, muscarinergic, or serotoninergic compound, and said receptor is respectively a histaminergic, dopaminergic, muscarinergic, or serotoninergic receptor.

311. The method according to claim 309, wherein said complement compound is selected from the group consisting of ascorbates, opioids, polycarboxylic acid chelators, resveratrol family members, cysteine family members, analogs and substituted derivatives thereof, and mixtures thereof.

312. A method for determining a regimen for regulating a biogenic amine receptor in a human or animal subject, comprising:
(a) selecting an aminergic compound useful for regulating said receptor, the aminergic compounds being a non-adrenergic aminergic compound or an adrenergic antagonist compound; and
(b) selecting a complement compound;
(c) determining the dosage level and frequency of dosing of said aminergic compound for use in regulating said receptor when administered to subjects in the absence of said complement;
(d) evaluating the effectiveness of said aminergic compound in regulating said receptor when administered to said subjects in the presence of said complement, as a function of the dosage level of said aminergic compound and the dosage level of said complement; and
(e) determining a regimen for regulating said receptor in said subjects by
(i) selecting a dose level of said aminergic compound which is determined to be effective in said evaluating step (d) and that is lower than the dosage level determined in said step (c);
(ii) selecting a dosage frequency that is determined to be effective in said evaluating step (d) and is longer than the dosage frequency determined in said step (c); or
(iii) both (i) and (ii).

313. The method according to claim 312, wherein said selecting step (b) comprises identifying said complement by use of at least one physical, chemical or immunological technique for detecting binding between said complement and said aminergic compound.

314. The method according to claim 312, wherein said aminergic compound is a histaminergic, dopaminergic, muscarinergic, serotoninergic, octopaminergic, or trace aminergic compound.

315. The method according to claim 312, wherein said complement is selected from the group consisting of ascorbates, opioids, polycarboxylic acid chelators, resveratrol family members, cysteine family members, analogs and substituted derivatives thereof, and mixtures thereof.

316. A pharmaceutical composition comprising:
(a) a subefficacious amount of an adrenergic antagonist compound; and
(b) a safe and effective amount of a complement compound.

317. The pharmaceutical composition according to claim 316, wherein said adrenergic antagonist compound is selected from the group consisting of afluzosin, buflomedil, bunazosin, corynanthine, dapiprazole, dihydroergocornine, dihydroergocristine, dihydroergocryptine, dihydroergotamine, dihydroergotoxine, doxazosin, ergotamine, ergotoxine, guanadrel, guanethidine, idazoxan, ifenprodil, indoramin, mianserin, mirtazapine, moxisylyte, naftopidil, nicergoline, phenoxybenzamine, phentolamine, prazosin, raubasine, reserpine, tamsulosin, terazosin, thymoxamine, tolazoline, trimazosin, urapidil, yohimbine; pharmaceutically acceptable salts or esters thereof; and combinations thereof.

318. The pharmaceutical composition according to claim 316, wherein said adrenergic antagonist compound is selected from the group consisting of acebutolol, alprenolol, atenolol, befunolol, betaxolol, bevantolol, bisoprolol, bopindolol, bucindolol, bucumolol, bufetolol, bufuralol, bunitrolol, bupranolol, butidrine, butofilolol, capsinolol, carazolol, carteolol, carvedilol, celiprolol, cetamolol, cloranolol, dilevalol, diprafenone, epanolol, ersentilide, esmolol, esprolol, indenolol, landiolol, levobunolol, medroxalol, mepindolol, metipranolol, metoprolol, moprolol, nadolol, nadoxolol, nebivolol, nifenalol, oxprenolol, penbutolol, pindolol, practolol, pronethalol, propafenone, propranolol, sotalol, sulfinalol, talinolol, tertatolol, tilisolol, timolol, toliprolol, trimepranol, xamoterol, xibenolol; amosulalol, arotinolol, labetalol, nipradilol; pharmaceutically acceptable salts or esters thereof; and combinations thereof.

319. The pharmaceutical composition according to claim 316, wherein said complement compound is selected from the group consisting of ascorbates, opioids, polycarboxylic acid chelators, resveratrol family members, cysteine family members, analogs and substituted derivatives thereof, and mixtures thereof.

320. The pharmaceutical composition according to claim 319, wherein said complement comprises an ascorbate.

321. A pharmaceutical composition comprising:
(a) a safe and effective amount of an adrenergic antagonist compound; and
(b) a complement compound selected from the group consisting of hyperpreserving amounts of ascorbates, analogs and substituted derivatives thereof; safe and effective amounts of morphines, analogs and substituted derivatives thereof; hyperpreserving amounts of polycarboxylic acid chelators, analogs and substituted derivatives thereof; hyperpreserving amounts of resveratrol family members, analogs and substituted derivatives thereof; safe and effective amounts of cysteine family members, analogs and substituted derivatives thereof; and mixtures thereof.

322. A method for treating a disorder associated with an adrenergic receptor in a human or animal subject, comprising:
(a) administering to said subject a safe and effective amount of an adrenergic antagonist compound; and
(b) administering to said subject a complement compound selected from the group consisting of: hyperpreserving amounts of ascorbates, analogs and substituted derivatives thereof; safe and effective amounts of opiates, analogs and substituted derivatives thereof; hyperpreserving amounts of polycarboxylic acid chelators, analogs and substituted derivatives thereof; hyperpreserving amounts of resveratrol family members, analogs and substituted derivatives thereof; safe and effective amounts of cysteine family members, analogs and substituted derivatives thereof; and mixtures thereof.
